(19) United States
(12) Patent Application Publication
(10) Pub. No.: US 2011/0040258 A1
(43) Pub. Date: Feb. 17, 2011

(54) Flexible and adjustable wrap for protecting and stabilizing intravenous catheter

(76) Inventor: Carroll V. Robison, Lindale, TX (US)

Correspondence Address: Carroll V. Robison, 14288 CR 434, Lindale, TX, 75771, US

(21) Appl. No.: 12/804,728

(22) Filed: Jul. 28, 2010

Related U.S. Application Data
(60) Provisional application No. 61/271,936, filed on Jul. 29, 2009.

(51) Int. Cl. A61M 25/02

(52) U.S. Cl. 604/179; 604/180

(57) ABSTRACT

A device contemplated to be used in lieu of tape to secure and protect an IV catheter while maintaining the patient's comfort and an acceptable range of movement is formed with a thin, flexible body configured to be wrapped around a portion of a patient's anatomy, and particularly a patient's hand. The body of the device includes a slot extending into the body from one end, with an aperture at the base of the slot into which a patient's thumb is to be received, and the ends of the device are wrapped around to the palm of the hand and secured to each other without any of the securing adhesive to contact the skin of the patient. In preferred embodiments a releasable and re-connectable cover is provided over the catheter site to allow inspection and adjustment with minimal disturbance of the device or the patient.

### RELATED APPLICATION DATA

This application claims the benefit of U.S. Provisional Patent Application Ser. No. 61/271,936, filed Jul. 29, 2009, by the same inventor/applicant.

### FIELD OF THE INVENTION

The present invention generally relates to medical devices, and in its preferred embodiments more specifically relates to devices for covering and securing a catheter, with associated tubing, inserted into a vein or artery in a human patient's arm, and especially the patient's hand, while maximizing freedom of movement and patient comfort. The invention includes devices for covering and protecting a catheter located on the patient's hand, and a catheter located on the patient's arm.

### BACKGROUND

It is a relatively common practice in the medical field to insert a catheter into a patient's vein or artery for the injection of fluids during a period of hospitalization, day surgery, etc. Since that practice began, one of the problems associated with it has been securing and stabilizing the catheter at the insertion site. Most commonly, a catheter is inserted into a vein. In the following description, the term “IV catheter” is used for convenience and is to be taken as encompassing both venous catheters and arterial catheters. The term catheter is used to refer to a tube, rigid or flexible, that is inserted through the patient's skin and into a vein, and a hub or connector that remains on the exterior of the skin, for connection of tubing to carry fluids through the catheter and into the patient. The most common and preferred insertion sites are the inside of the patient's arm, and the back of the patient's hand. Unless the exterior portion of the catheter is well secured, it can be easily pushed or pulled out of position, which can result in full or partial removal of the catheter, usually accompanied by both physical and mental distress for the patient. Inadvertent removal of the catheter from the vein or from the patient completely may cause injury to the patient, and, at the least, will interfere with the administration of fluid through the catheter, potentially creating a life-threatening situation.

In normal current practice the catheter is inserted into a vein and then secured by applying a sufficient amount of adhesive tape over the catheter on the patient's arm to assure that the catheter remains in place and is not disturbed by normal movements. Typically, a medical practitioner will apply a substantial amount of tape over the catheter and over tubing running from the catheter. That practice is time consuming, wasteful of tape, and, perhaps most significantly, uncomfortable for the patient. After a catheter is inserted it must be periodically inspected, and the insertion site may be changed, both of which require removal of some or all of the tape. Removal of the tape can be considerably more uncomfortable than placement. Some patients are allergic or otherwise sensitive to the adhesive, and those patients may suffer skin burns and other adverse reactions.

Various efforts have been made to alleviate the problems associated with stabilizing and protecting an IV catheter, and a number of devices are known in the prior art. Some of these devices provide very secure confinement of an IV catheter and associated tubing, but also restrict patient movement and compromise comfort. Others are more comfortable to the patient, but provide less effective stability for the catheter apparatus. None of the known devices from the prior art have been fully successful in addressing the need for a device that provides both acceptable catheter security and patient comfort, and the use of adhesive tape remains the most prevalent manner of securing a catheter, despite the well known problems associated with that approach.

### SUMMARY OF THE INVENTION

The present invention provides a device to be used in lieu of tape to secure and protect a catheter while maintaining the patient's comfort and an acceptable range of movement. The device is preferably formed of a flexible, non-woven, tear-resistant fabric material in a generally rectangular configuration. An opening is formed in the piece of material and covered with a flexible transparent panel or window that is adhered to the material around the opening. In a particularly preferred embodiment, the panel may be lifted to allow care giver access to the catheter, and then re-affixed to again establish the protective cover over the catheter without replacing the complete device each time access is required. The device of the invention will be provided in at least two configurations, one to secure a catheter placed on the back of the patient's hand, and another to secure a catheter placed on the arm adjacent to the elbow. The structure and features of preferred embodiments of the device will be described in detail below, with reference to the accompanying drawing figures.

### BRIEF DESCRIPTION OF THE DRAWINGS

FIG. 1 is a top plan view of a preferred embodiment of a hand wrap configuration of the device of the invention, for use on a patient's left hand.

FIG. 2 is a side elevation view of the hand wrap embodiment of the device of FIG. 1, looking toward the first edge of the device, in which the thickness of the components of the device is exaggerated for clarity.

FIG. 3 is a top plan view of a particularly preferred embodiment of the hand wrap device configuration of FIG. 1.

FIG. 4 is a top plan view of the embodiment of the device as in FIG. 3, with a removable and adjustable catheter cover.

FIG. 5 is a side elevation view of the hand wrap embodiment shown in FIG. 4 looking toward the first edge of the device, in which the thickness of the components is exaggerated for clarity.

FIG. 6 is a top plan view of the hand wrap configuration embodiment of FIG. 1, in place upon the back of a patient's hand, with a catheter in place, and ready for connection.

FIG. 7 is a top plan view Of the hand wrap embodiment of the device of FIG. 1, with the first end of the device wrapped around to the palm of the hand in one of the steps for connection of the device around the hand.

FIG. 8 is a top plan view of a particularly preferred embodiment of hand wrap configuration of the device, in progress of attachment in the position and manner shown in FIG. 7 for that embodiment.

FIG. 9 is a top plan view of the hand wrap embodiment of the device configuration of FIG. 1, with both ends of the device wrapped around to the palm of the hand and connected to each other to secure the device around the hand and over a catheter.

FIG. 10 is a top plan view of the particularly preferred embodiment of the hand wrap configuration of, with both ends of the device wrapped around the hand as in FIG. 9.

FIG. 11 is a top plan view of an embodiment of the hand wrap configuration of the device and the palm of a patient's hand, showing the configuration of the connected device on the palm side of the hand, the palm side view being the same for all embodiments.

FIG. 12 is a is a top plan view of the an embodiment of the hand wrap configuration of the device of the invention, for use on a patient's right hand.

FIG. 13 is a top plan view of an embodiment of the arm wrap configuration of the device of the invention.

FIG. 14 is a side elevation view of the embodiment of the arm wrap configuration shown in FIG. 13, looking toward the first edge of the device, in which the thickness of the components is exaggerated for clarity.

FIG. 15 is a top plan view of a particularly preferred embodiment of the arm wrap configuration of the device.

FIG. 16 is a top plan view of a variation of the particularly preferred embodiment of the arm wrap configuration of the device.

FIG. 17 is a top plan view of an embodiment of the arm wrap configuration of the device, laid across the inside of a patient's arm in preparation for connection of the device around the arm.

FIG. 18 is a is a top plan view of the embodiment of the arm wrap configuration of the device, as in FIG. 17, positioned over the arm, with the first end of the device wrapped around the arm.

FIG. 19 is a top plan view of the embodiment of the arm wrap configuration of the device of FIG. 17, positioned over the arm, with both ends wrapped around the arm for connection to each other.

FIG. 20 is a top plan view of a particularly preferred embodiment of the arm wrap configuration, with both ends wrapped around the arm, allowing access directly to the catheter insertion site without fully removing the device.

FIG. 21 is a top plan view of an elongate strip for wrapping around a patient's arm to retain tubing extending from a catheter insertion site to a source of IV fluid or the like.

FIG. 22 is an edge elevation view of a first embodiment of an elongate retaining strip with an adhesive strip at both ends, connected to a section of tubing (shown in cross-sectional view).

FIG. 23 is an edge elevation view of a second embodiment of an elongate retaining strip with an adhesive strip at one end, connected to a section of tubing (shown in cross-sectional view).

### DESCRIPTION OF THE INVENTION

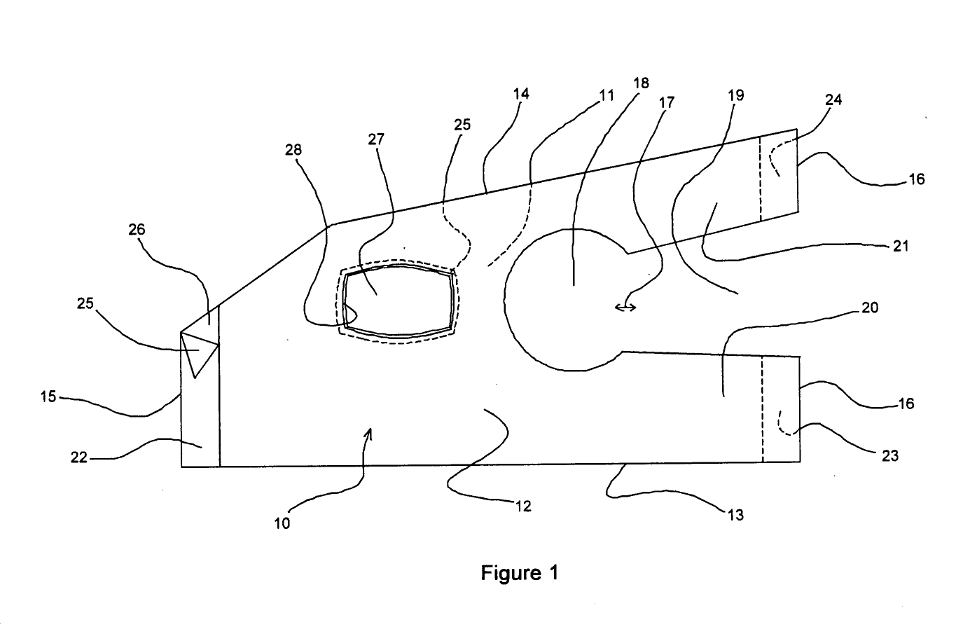

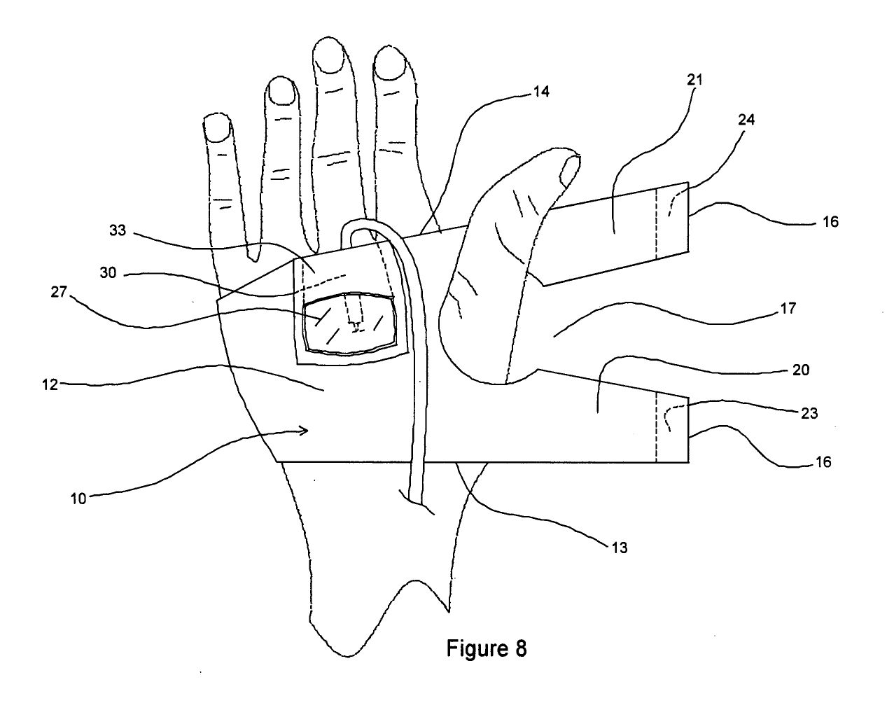

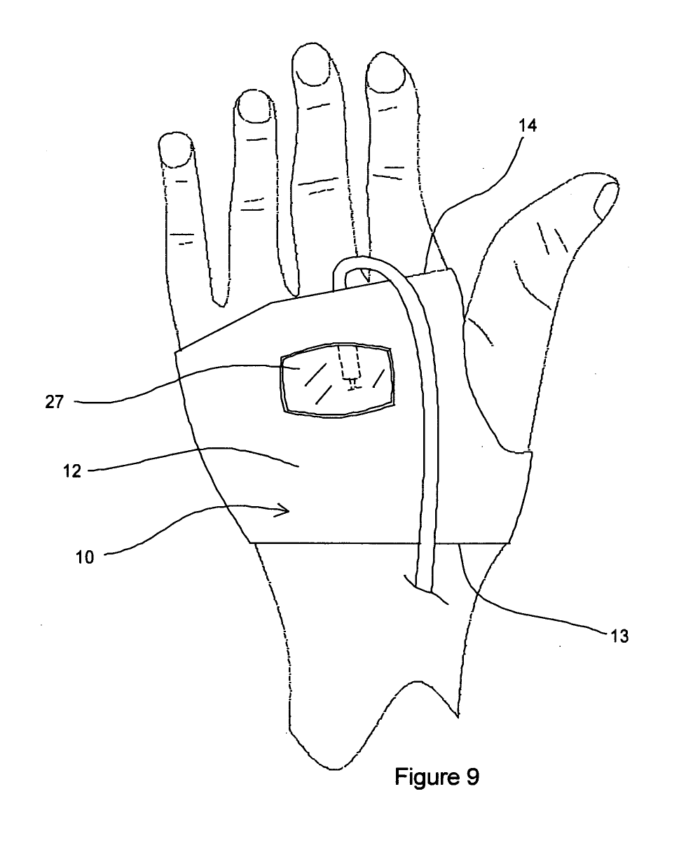

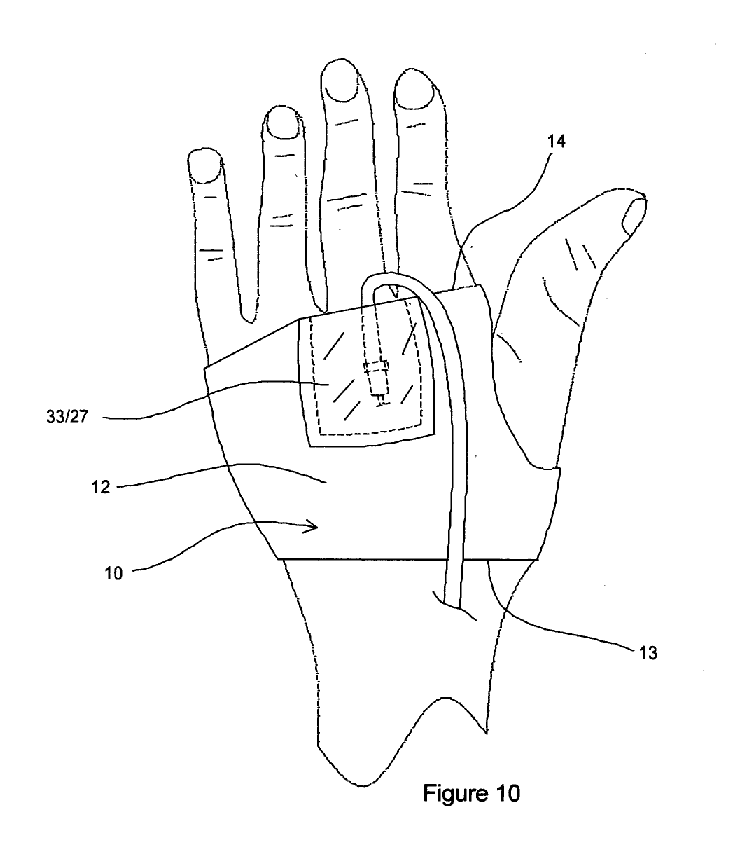

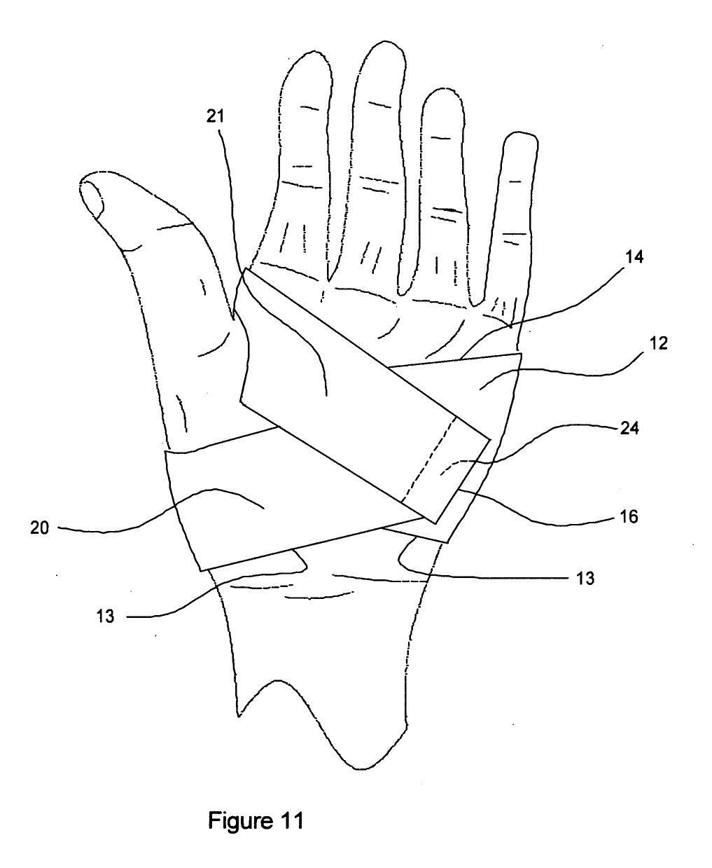

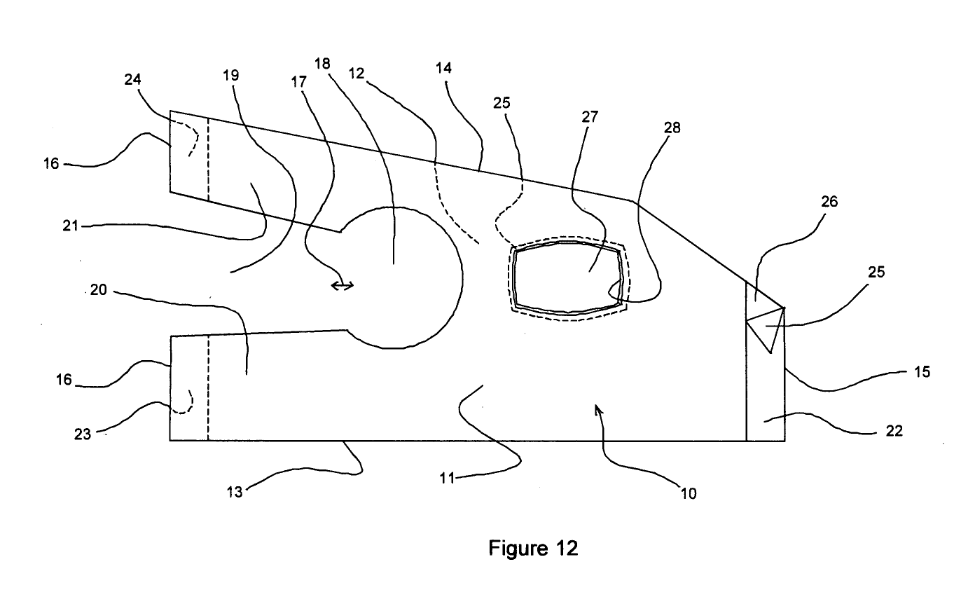

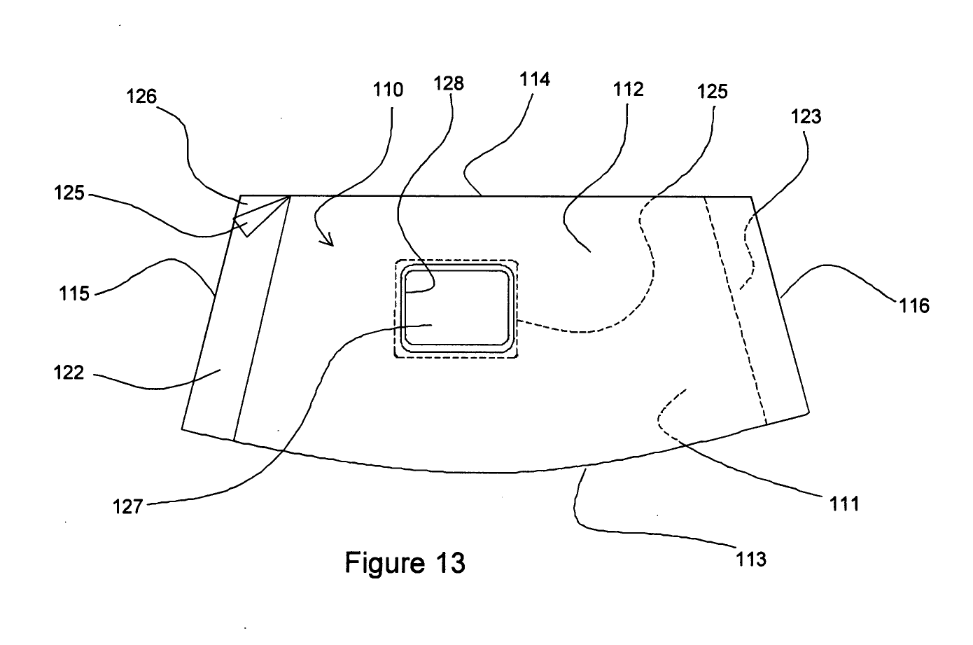

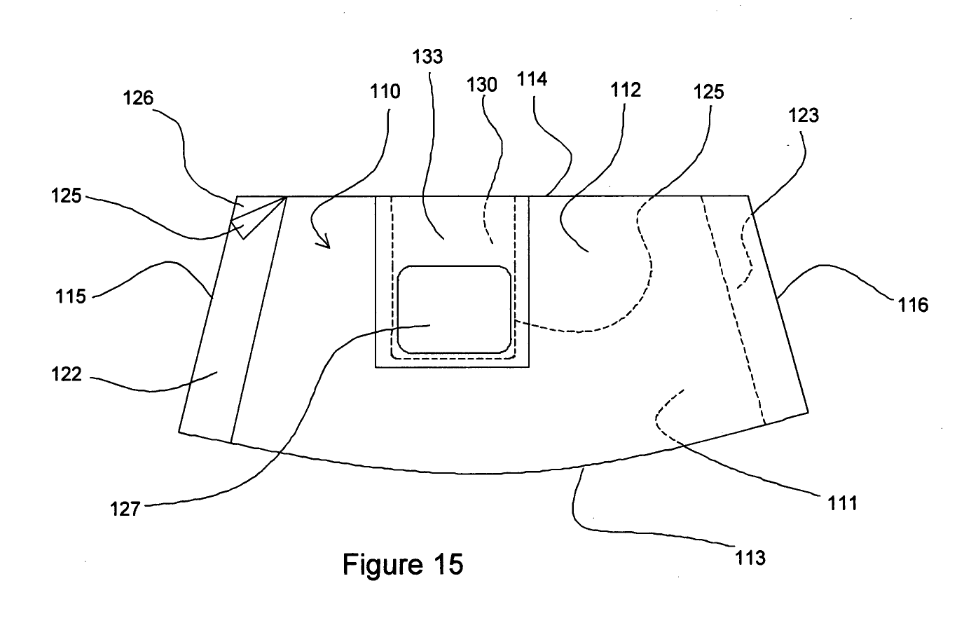

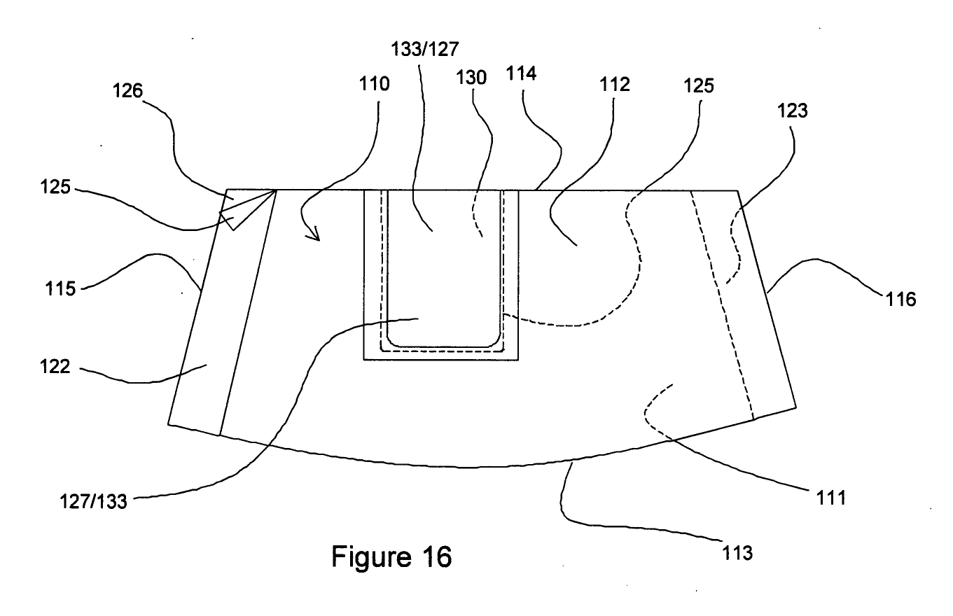

The device of the invention is contemplated to be provided in at least two configurations, each having at least two embodiment; a first, or hand wrap, configuration to be used to secure an IV catheter placed in a vein on the back of the patient's hand adjacent to the wrist, illustrated in FIGS. 1-12, and a second, or arm wrap, configuration to secure a catheter placed in a vein on the inside of a patient's arm, illustrated in FIGS. 13-20.

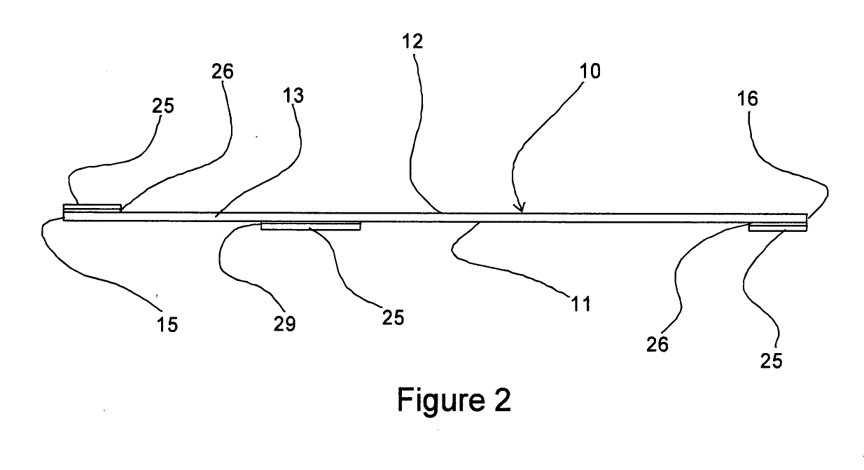

Referring first to FIGS. 1 and 2, a first, or basic embodiment of the hand wrap configuration of the invention is formed as a thin, flexible body 10, with a first face 11, a second face 12, a first edge 13, a second edge 14, a first end 15, and a second end 16. In the embodiment shown, first edge 13 is generally straight, and second edge 14 is formed at an angle to the first edge, so that the width of body 10 between the first and second edges increases between first end 15 and second end 16. Second edge 14 is preferably curved along its length to follow the shape of a patient's hand during placement and use of the device, but it is to be understood that the scope of the invention is not limited to the proportional relationships specifically shown and described, but encompasses a variety of proportional relationships, so long as the selected proportional relationship provides acceptable fit and function.

In this embodiment body 10 includes a generally keyhole-shaped cut-out 17 formed in body 10, the keyhole including a generally circular aperture 18 disposed between the first and second edges and the first and second ends, and a relatively wide slot 19 extending from the second end of the body to aperture 18. Removing a portion of body 10 to form cut-out 17 and slot 19 creates a first strip 20 and a second strip 21, separated by slot 19. Strip 20 includes the portion of the body along first edge 13 between that edge and the adjacent edge of cut-out 17, and a portion of second end 16 of the body. Strip 21 includes the portion of the body along second edge 14 and the adjacent edge of cut-out 17 and a portion of second end 16. Cut-out 17 is preferably formed with smooth transitions at the intersection of slot 19 with aperture 18, but the exact nature of the transitions is not critical.

So that the device may be easily and releasably applied to a patient, the described and illustrated embodiments include a first adhesive band 22 on face 12 of body 10 along first end 15, and extending from the end of the device a short distance into the interior toward the second end. A second adhesive band 23 is formed on face 11 of body 10 along the second end portion of strip 20, and a third adhesive band 24 is formed on face 11 of the body along the second end portion of strip 21. The width of the adhesive bands from the end of the body inward is preferably approximately one inch, but any width that will assure secure adhesion to the material from which the body of the device is formed may be satisfactorily used. It is preferred that the adhesive material forming the adhesive bands be covered with a release sheet 25 for protection. The same adhesive material 26 is preferably used to form bands 22, 23, and 24 so that the adhesive strength between each band and the body of the device is the same.

The device of the invention further includes a transparent window 27 disposed in body 10 between aperture 18 of cut-out 17 and the first end 15 of the body, covering a window aperture 28 formed in body 10. Window 27 is formed of a flexible transparent material, and will cover the insertion site of a catheter through the patient's skin to provide stability and protection for the catheter while allowing the insertion site and catheter to be visually inspected. It is preferred that the face of window 27 to be received against the skin of a patient over the catheter insertion site be covered with a transparent adhesive 29 that is gently adhesive to human skin, to facilitate placement and retention of the device with window 27 over the catheter insertion site for visibility. Alternatively, adhesive compound 29 may be provided around the perimeter of the window to provide such adhesion without adhering the window itself to the catheter. A preferred type of adhesive material for window 27 is non-irritating to human skin and forms a bond to skin that is easily removed without damaging the skin, but it is to be understood that adhesive 29 could be omitted from the window or window perimeter if desired.

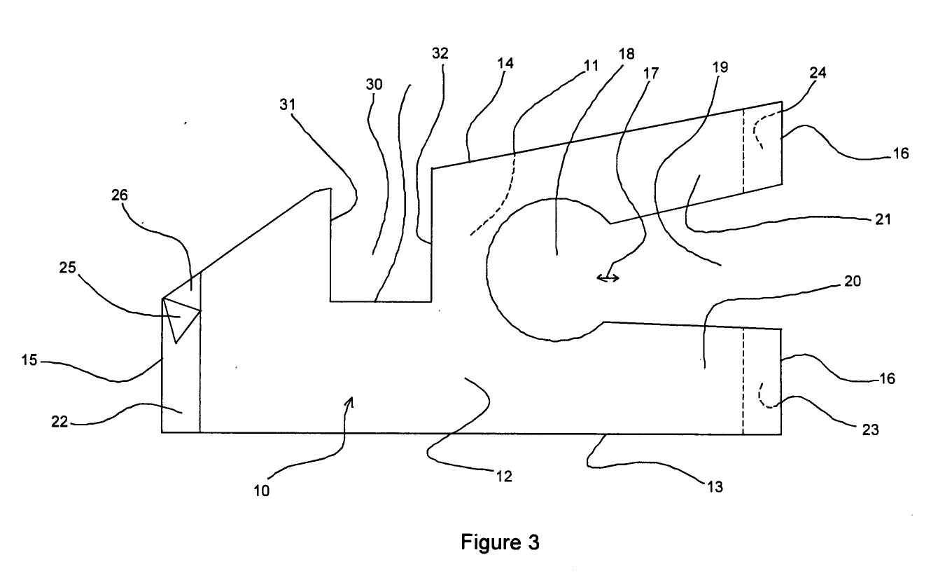

In a particularly preferred embodiment, shown in FIGS. 3-5, window aperture 28 is replaced by a slot 30 formed in body 10 to extend inwardly from second edge 14 of the body a distance generally equal to the distance between edge 14 and the farthest edge of aperture 28 from edge 14 in the previously described embodiment. Slot 30 has first and second parallel opposed edges 31 and 32. Instead of a discrete window panel 27 adhered at its edges to body 10 around aperture 28, a catheter cover panel 33 is disposed over slot 30 and releasably adhered along three of its edges to body 10. Cover panel 33 includes opposed side edges 34 and 35 and opposed inner and outer edges 36 and 37, and is larger in dimension than slot 30 so that the side edges and the inner edge 36 overlie a portion of body 10 around slot 30 and are received against the second face 12 of body 10 around the edges of the slot. In one variation, cover panel 33 may be formed of the same transparent material as window panel 27, to permit visual inspection of the entirety of the catheter and tubing that underlies the device when in place on a patient's hand. When access to the catheter and/or tubing is required, panel 33 can be detached and lifted at least partially away from the catheter and tubing to provide access, and then re-adhered, allowing the same device to remain in place rather than being replaced each time catheter access is needed.

In a variation, a window 27, essentially as described above, may be structured within a portion of cover 33, with the remainder of cover 33 formed of the same material as body 10. As noted above, the skin-contact side of the transparent window material, or of the entire cover panel 33 if desired, may be provided with an adhesive layer to gently adhere it to the patent's skin over the catheter and connected tubing, or may be left adhesive free.

A variety of materials could be used to form body 10 of the device within the scope of the invention, but it is preferred that the material of construction have certain properties or characteristics that are deemed to be desirable. It is preferred that the material be of a woven fabric construction, breathable, and hypo-allergenic. Other desirable but non-essential characteristics include slight deformability without tearing, and some degree of elasticity. It is to be understood that any suitable material that will provide appropriate performance may be used within the scope of the invention.

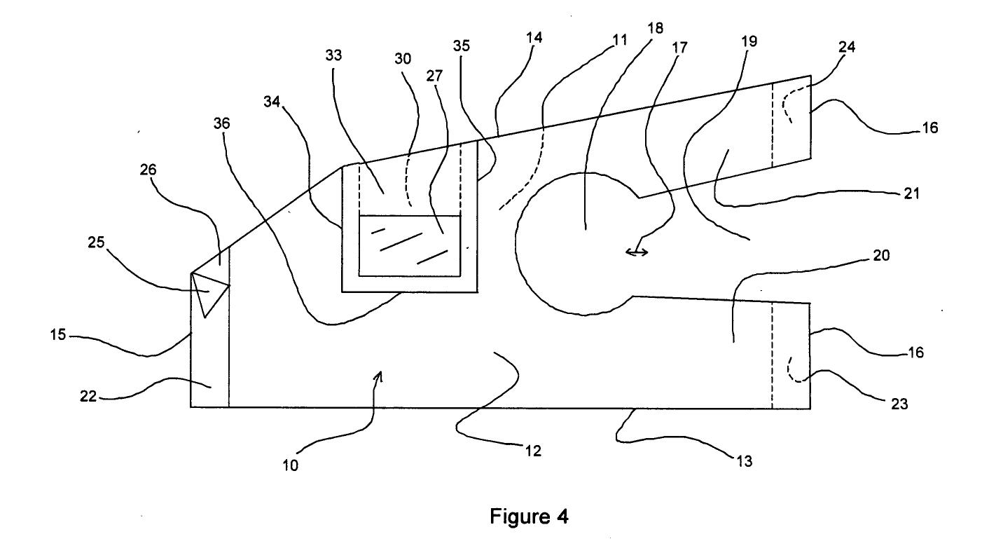

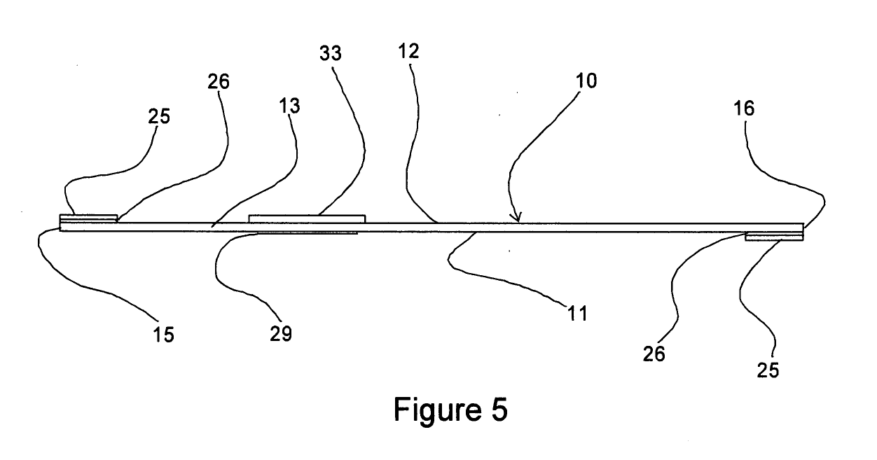

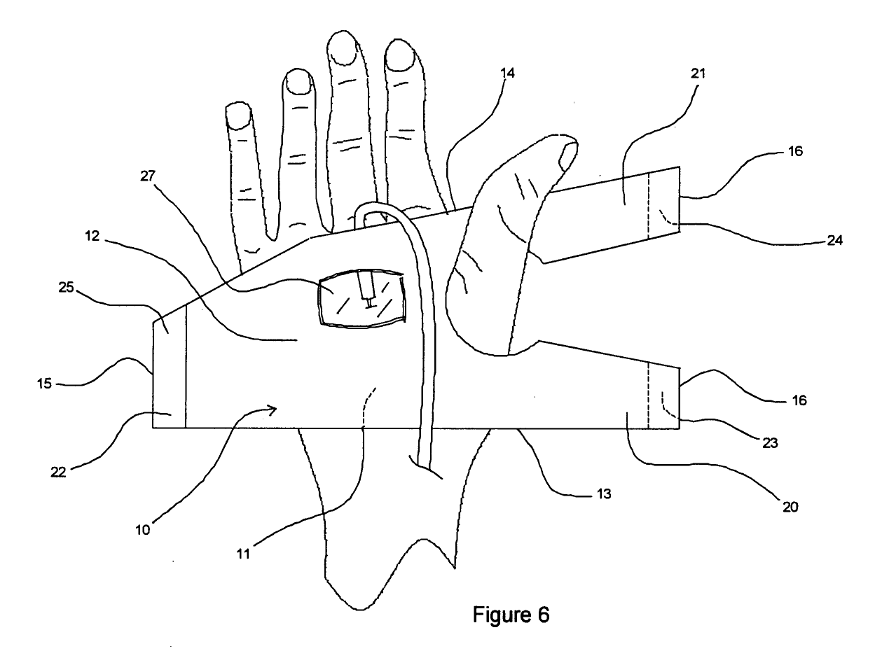

To use the device of the invention to secure and protect a catheter inserted through a patient's skin on the back of the patient's left hand adjacent to the wrist, the device of the invention is, as illustrated in FIG. 3, placed on the back of the patient's hand with face 11 in contact with the skin and with window 27 directly overlying the catheter insertion site, after first removing the release sheet 25, if adhesive is used on or around the window, and the window is lightly pressed against the patient's skin around the catheter. The first end of the device extends outwardly beyond the edge of the hand opposite the thumb, and strips 20 and 21 extend outwardly beyond the opposite edge of the hand, with the thumb between the strips. The first end of the device is wrapped over the edge of the hand opposite the thumb and over a portion of the palm of the hand, as shown in FIG. 4, with first adhesive band 22 facing upward, away from the hand. Release sheet 25 is removed from first adhesive band 22, and the release sheets are also removed from the second and third adhesive bands 23 and 24. First band 20 is wrapped around the heel of the patient's hand and across the palm, over adhesive band 22 at the first end of the device, and onto the body of the device. Adhesive band 22 engages the facing surface of strip 20, and adhesive band 23, at the end of strip 20, engages the body of the device it overlies. Strip 21 is then wrapped around the opposite side of the thumb between the thumb and forefinger and onto the body of the device overlying the palm. In the same manner as with strip 20, strip 21 adheres to adhesive band 22, and the third adhesive band 24 adheres to the body of the device, securing the device in place around the patient's hand and over the catheter insertion site, as can be seen in FIG. 5. FIG. 6 shows the device connected to the hand as in FIG. 5, from the palm side of the hand. Strip 21 will typically overlie strip 20, as shown, to achieve a close fit of the device on the patient's hand, but it is to be understood that the specific placement and/or order of placement of strips 20 and 21 is not critical and can be varied, so long as a secure fit is achieved.

It will be understood from the foregoing that none of the adhesive forming adhesive bands 22, 23, and 24 is brought into contact with the patient's skin, eliminating the possibility of skin irritation or a more severe allergic reaction to the adhesive. The only adhesive in direct contact with the patient's skin is the adhesive associated with window 27 and/or panel 33. Because the adhesive bond between the window and the skin plays a limited role on retaining the device on the hand, a mild adhesive, much less likely to cause an adverse reaction, may be used, or, as noted above, adhesive may be omitted from the window altogether. The configuration of the device and its placement on the hand, with strips 20 and 21 on opposite sides of the thumb, prevents the device from sliding forward toward the fingers, backward toward the wrist, and/or around the hand, and thus provides a secure protective covering for a catheter and its insertion site while avoiding the disadvantages and discomfort associated with taping the catheter in place.

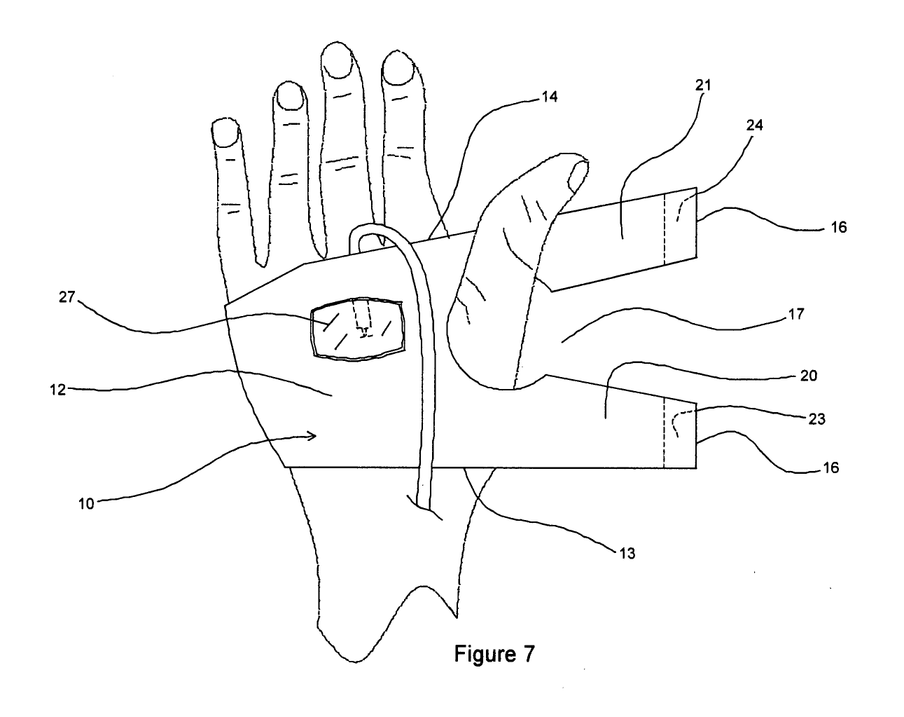

The body of the device, absent the adhesive bands at the first and second ends of the body and absent the adhesive associated with window 25, can be used for either the left hand or the right hand. The only difference between a left-hand version and a right-hand version is the face of the body on which the adhesive bands, and window adhesive, are placed. For a right-handed version, shown in FIG. 7, adhesive band 22 is formed on first face 11 of the body rather than on second face 12, and adhesive bands 23 and 24 are formed on second face 12. Adhesive for window 27 is placed on or around the face of the window that will be in contact with the skin.

The hand wrap configuration of the device may also be made universal, or reversible, in a variation that may be used on either hand interchangeably. Looking to FIG. 7, in a universal variation of the device, an adhesive band 22 is formed on both first and second faces of body 10 at first end 15, each covered by a release strip 25. The adhesive material for window 25 may be placed on both sides of the window, each covered by a release sheet, or window adhesive may be omitted altogether. In one embodiment of this variation, adhesive material is omitted entirely from the ends of strips 20 and 21, and the device in connected to a patient by first wrapping strips 20 and 21 around to the palm of the hand and then wrapping the first end 15 around to overlie and connect to the strips. In another embodiment, adhesive material may be placed on both faces of the ends of strips 20 and 21, like the doubled adhesive band described above, with each layer of adhesive material covered by a release sheet. When the device is connected to a patient, the care giver selects which sets of release strips to remove so as to accommodate the selected one of the patient's hands, and the device is connected as described above for the non-reversible embodiments.

An arm wrap configuration of the device of the invention, shown in FIGS. 13-20, includes a body 110, a first face 111, a second face 112, a first edge 113, a second edge 114, a first end 115, and a second end 116. It is preferred that second edge 114 be generally straight, and that first edge 113 be slightly curved between the first and second ends of the body, and be of greater length than second edge 114. First and second ends 115 and 116, extend between the first and second edges of the body at a non-perpendicular angle relative to either edge.

To provide for connection of the arm wrap configuration around the arm of a patient, a first, or basic, embodiment includes a first adhesive band 122 on face 112 of body 110 along first end 115, and extending from the end of the device a short distance into the interior toward the second end. A second adhesive band 123 is formed on face 111 of body 110 along second end 116. As in the hand wrap embodiments, the width of the adhesive bands from the end of the body inward is preferably approximately one inch. It is also preferred that the adhesive material forming all adhesive bands be covered with a release sheet 125. The same adhesive material used for the bands 22, 23, and 24 of the hand wrap embodiment is also preferably used for bands 122 and 123 in the arm wrap embodiments, and is identified by reference number 126 in the context of the arm wrap embodiment.

The first arm wrap embodiment also includes a transparent window 127 disposed in body 110 over a window aperture 128. The description of window 27, above, is equally applicable to window 127 of the arm wrap configuration, and need not be repeated here. As in the hand wrap embodiment, it is preferred that window 127 be directly adhesive to the patient's skin or be surrounded by adhesive material 129. The adhesive material, preferably the same as adhesive 29, the nature of which is as described above, may be omitted from the window or the surrounding area of the body if desired, also as discussed above, within the scope of the invention. If used, window adhesive 129 is disposed on the face of window 127 that is associated with the first face 111 of the body and is to be received against the patient's skin. Window 127 is preferably not disposed in the geometric center of body 110, but is offset toward edge 114 and toward end 115 from the center. The off center placement of window 127 is preferred in order to facilitate placement and connection of the device of the invention around a patient's arm, but is not an essential feature, and other proportional relationships may be used.

In a particularly preferred embodiment of the arm wrap configuration, window aperture 128 is replaced by a slot 130, with edges 131 and 132, that extends into body 110 from edge 114, and is covered by a cover panel 133. As described above, the entire cover panel 133 may be formed of a transparent window material, or may be formed of the material of body 110 with a window panel 127 disposed within it.

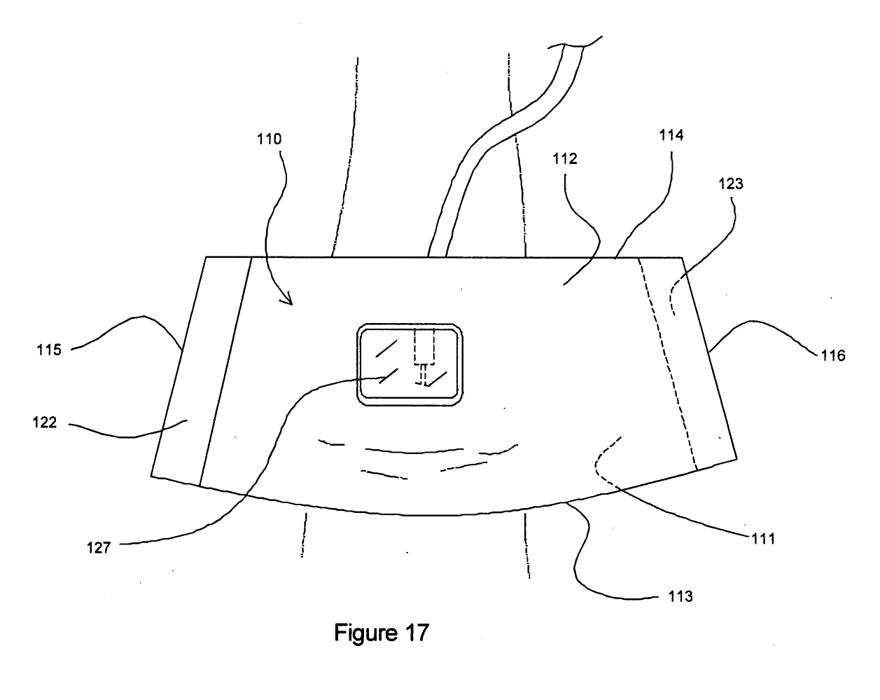

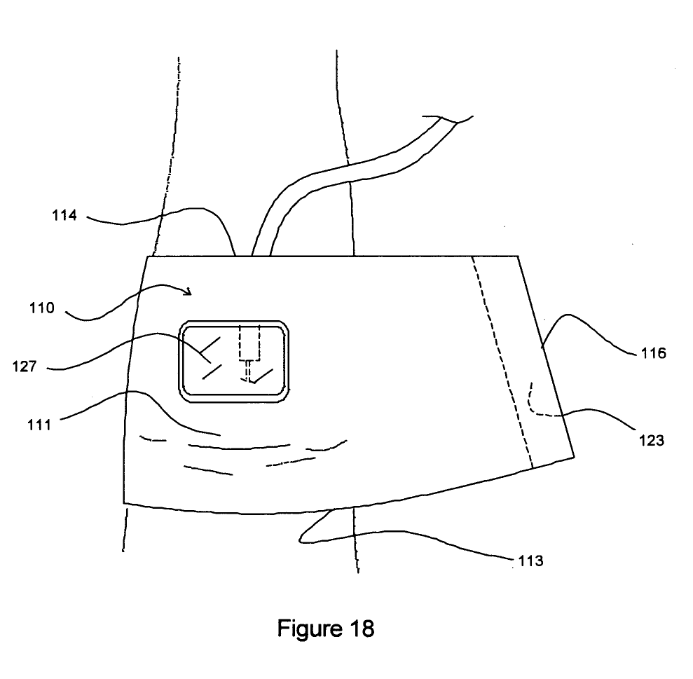

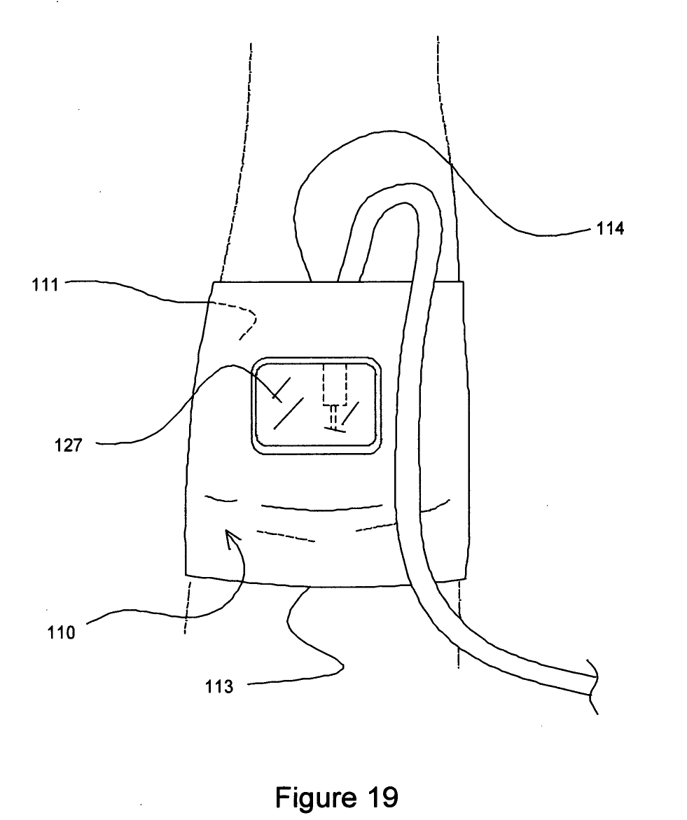

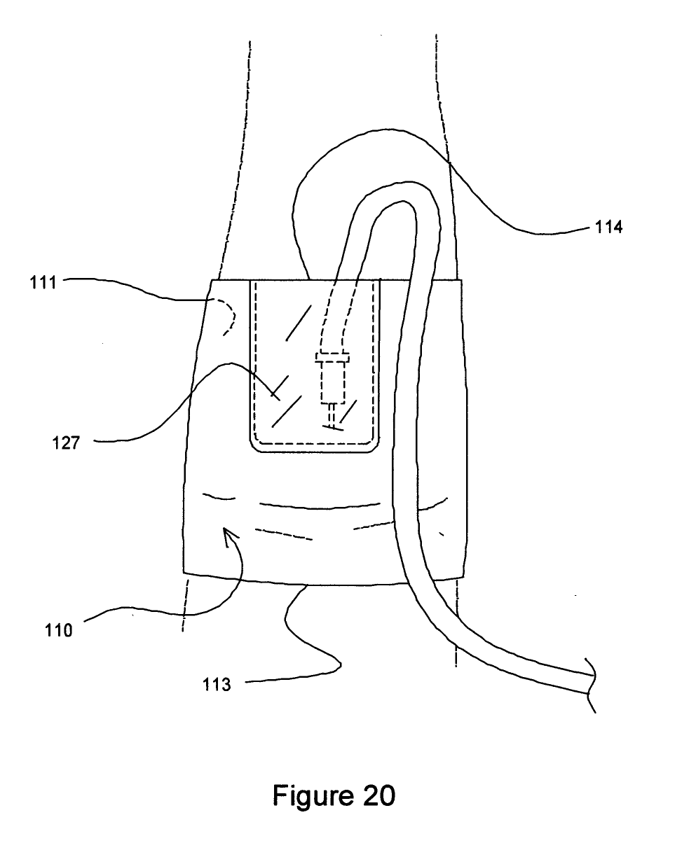

As illustrated in FIG. 17, the arm wrap embodiment of the device of the invention is used to cover and secure a catheter inserted into a patient's arm adjacent to the elbow by first removing the release sheet 125 covering the window adhesive 129, if used, and placing the device over with catheter with window 127 overlying the catheter, so that the catheter is visible through the window material. The device is oriented on the patient's arm with edge 114 facing the wrist, and with edge 113 facing or slightly overlapping the inside of the elbow. The release sheets 125 are removed from adhesive bands 122 and 123. The first end 115 of the body of the device is wrapped around the patient's arm and received against the patient's skin, with adhesive of band 122 on the surface of the device facing away from the patient's skin, as in FIG. 18. The first end of the device is held in place while the second end 116 is wrapped in the opposite direction until the portion of the body at the second end overlaps the first end of the body, bringing the portion of the body adjacent to the second end into contact with adhesive band 122, and bringing adhesive band 123 into contact with the portion of the body adjacent to the first end. FIGS. 19 and 20 show different embodiments of the device fully wrapped around the arm. The adhesive bonds formed between the adhesive material and the material of the body retains the device around the patient's arm without contact between the material of adhesive bands 122 and 123 and the patient's skin.

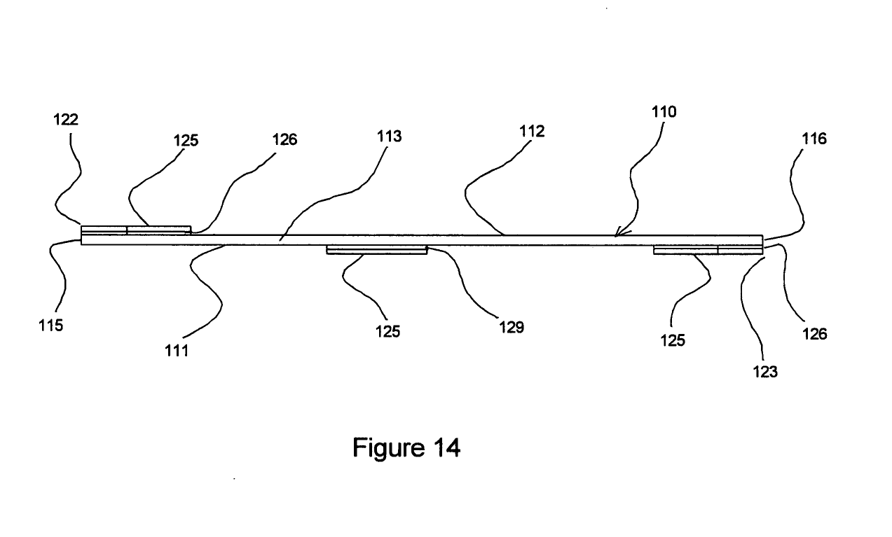

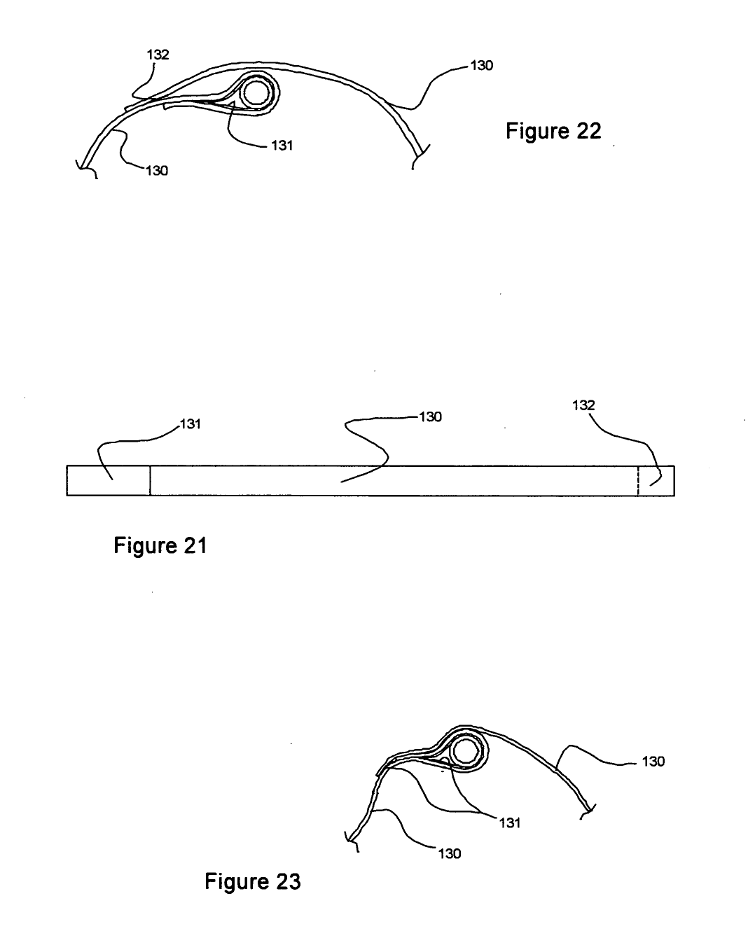

Elongate strips 134 of the same material as body 10 or 110, with one or more adhesive bands, may be provided with the primary device of the invention in a kit, and used to secure IV tubing routed along a patient's arm. Connection of these strips around the arm is preferably accomplished in generally the same manner as body 110 is connected, so as to avoid direct contact between the adhesive and the patient's skin. An embodiment with adhesive bands 135 and 136 at opposite ends of strip 134 is illustrated in FIGS. 21 and 22. When such strips are used, adhesive band 135 at one end of the strip may be wrapped around and adhesively secured to the tubing, the strip is then wrapped around the patient's arm, and adhesive band 136 at the opposite end is connected to the strip itself, as illustrated in FIG. 14. Alternatively, as shown in FIG. 23, adhesive band 136 may be omitted and band 135 made with sufficient length to wrap around the tubing with a length of adhesive exposed to receive and adhere to the opposite end of the strip. In both embodiments of strip 134, the same adhesive material used to form adhesive bands 122 and 123 is preferably used for bands 136 and/or 135, although other adhesive materials could be used if desired.

With either embodiment of the device in place and secured on a patient's hand or arm, the catheter and insertion site are covered and protected by the transparent window, which allows the catheter to be seen, and/or to be seen and adjusted, depending on the embodiment chosen, without removing or rearranging the device. The catheter is retained in place between the device and the patient, the IV tubing adjacent to the catheter can also be held in place by the device, and can be, if desired, secured to the outer surface of the device, rather than to the patient's skin, with tape.

With the device of the invention, nothing more irritating than the mild window adhesive, if that, is in direct contact with the patient's skin, and most of the discomfort associated with IV catheters is avoided. While the device is in place, air can reach the patient's skin where tape would otherwise cover it, and the device can move slightly to accommodate patient movements without pulling on the skin. When the device is removed, the patient's skin and/or hair is not pulled, no adhesive residue is left, and no adhesive induced skin irritation is created. Therefore, the device can be removed and replaced easily and quickly whenever replacement is warranted, without creating concern for the patient's skin condition and comfort.

The foregoing description of preferred embodiments, alternatives, and variations of the device of the invention is intended to be illustrative and not limiting. It is contemplated that further embodiments and variations may be devised from and on the basis of the present invention, the scope of which is to be understood in accordance with the following claims.

1. A device for securing and protecting an IV catheter, hub, and immediately adjacent tubing inserted into the back of a hand of a human patient, fitted in such a way as to minimize patient discomfort and maximize hand and finger mobility and dexterity, and structured so as to prevent direct contact between adhesive used to secure the device and the skin of the patient, comprising,
a flexible body, formed of a thin pliable material, said body having a first face and a second face opposed to said first face across the thickness of said body, a first edge and a second edge generally opposed to said first edge, a first end and a second end generally opposed to said first end, said body being of a sufficient length between said first and second ends to extend across the width of the back of a human hand to which the device is to be connected with said first end of said body extending outwardly from the edge of the hand adjacent thereto, with said second end of said body extending outwardly from the opposite edge of the hand, the distance of extension of said first end of said body from the adjacent edge of the hand being less than the width of the hand between the two edges of the hand, and the distance of extension of said second end of said body from the opposite edge of the hand being less than the width of the hand between the two edges of the hand;
said body having a cut-out formed therein, said cut-out including a generally rounded thumb aperture disposed between said first and second edges and between said first and second ends of said body, and a slot extending into said body between said second edge and said thumb aperture, within which slot the thumb of a patient's hand is to be received, forming a first strip including the portion of said body along said first edge between said first edge and the adjacent edge of said slot, and a portion of said second end of said body, and a second strip including the portion of said body along said second edge between said second edge and the adjacent edge of said slot, and the remaining portion of said second end of said body;
said body further including a first adhesive band disposed on said second face of said body at said first end thereof, said first adhesive band extending across said first end between said first and second edges and inward a short distance toward said second end of said body, said first band of adhesive disposed such that when said body of the device is placed over the back of a patient's hand with said first face received against the skin of the hand and said first end of said body is wrapped around the adjacent edge of the hand to the palm of the hand said first adhesive band is disposed on said face of said body that is positioned outward of the skin of the hand, preventing said first adhesive band from contacting the skin of the hand;
said body further including a second adhesive band disposed on said first face of said body at said second end of said first strip, said second adhesive band extending across the outer end of said strip at said second end of said body between said first edge of said body and said slot, said second band of adhesive disposed such that when said first strip is wrapped around the adjacent edge of the hand on one side of the thumb to the palm of the hand, said first strip overlies said first end of said body, said second adhesive band is brought into contact with said second face of said first end to form an adhesive connection between said second adhesive band and said second face of said first end, and said first adhesive band is brought into contact with said first face of said first strip to form an adhesive connection between said first adhesive band and said first face of said first strip without either adhesive band contacting the skin of the patient; and
said body further including a third adhesive band disposed on said first face of said body at said second end of said second strip, said third adhesive band extending across the outer end of said second strip at said second end of said body between said second edge of said body and said slot, said third band of adhesive disposed such that when said second strip is wrapped around the adjacent edge of the hand on the opposite side of the thumb as said first strip, to the palm of the hand, said second strip overlies said first end of said body and overlies said first strip connected thereto, said third adhesive band is brought into contact with said second face of said first strip to form an adhesive connection between said third adhesive band and said second face of said first strip without any of said adhesive bands contacting the skin of the patient.

2. The device of claim 1, further comprising a window aperture formed in said body between said first and second edges and said first and second ends, said aperture positioned in said body so as to be disposed over the site of penetration of an IV catheter through the skin of a patient on the back of the patient's hand with the device connected around the patient's hand, so as to permit visual inspection of the catheter without removing the device from the patient.

3. The device of claim 2, further comprising a window cover formed of a transparent material, said cover connected to said body around said window aperture so as to provide a protective barrier to penetration through said window aperture while allowing visual inspection of the catheter without removing the device from the patient.

4. The device of claim 1, wherein said first adhesive band is disposed on both said first face and on said second face of said body at said first end thereof, wherein said first adhesive band on said first face is covered by a selectively removable release sheet and said first adhesive band on said second face is covered by a separate selectively removable release sheet, and wherein said second adhesive band and said third adhesive band are omitted.

5. The device of claim 1,
wherein said first adhesive band is disposed on both said first face and on said second face of said body at said first end thereof, and wherein said first adhesive band on said first face is covered by a selectively removable release sheet and said first adhesive band on said second face is covered by a separate selectively removable release sheet;
wherein said second adhesive band is disposed on both said first face and on said second face of said body at said second end of said first strip, and wherein said second adhesive band on said first face is covered by a selectively, removable release sheet and said second adhesive band on said second face is covered by a separate selectively removable release sheet;
wherein said third adhesive band is disposed on both said first face and on said second face of said body at said second end of said second strip, and wherein said third adhesive band on said first face is covered by a selectively removable release sheet and said third adhesive band on said second face is covered by a separate selectively removable release sheet.

6. The device of claim 3, wherein said window cover includes an adhesive material applied to the lower face of said window cover to be brought into contact with the patient's skin around the catheter and to form a mild and easily separable adhesive bond between said window cover and the skin of the patient.

7. The device of claim 1, further comprising a first release sheet temporarily covering said first adhesive band until removed, a second release sheet temporarily covering said second adhesive band until removed, and a third release sheet temporarily covering said third adhesive band until removed, so as to prevent premature exposure of said adhesive bands and unintended adhesion of any of said bands to an undesired surface or in an undesired location.

8. The device of claim 1, further comprising,
a catheter inspection slot extending into said body from said first edge of said body toward said second edge, between said first end of said body and the inner extension of said thumb aperture, said inspection slot disposed and dimensioned so as to overlie the penetration site of a catheter on the back of a patient's hand; and
an inspection slot cover formed of a transparent material, said inspection slot cover releasably connected to said body around said inspection slot, to be positioned over the catheter insertion site, so as to allow visual inspection of the catheter without removing either the inspection slot cover or the complete device from the patient, and to allow said cover to be lifted to provide physical access to the catheter and then re-adhered to re-establish protection of the catheter without removing and replacing the entire device.

9. The device of claim 1, further comprising,
a catheter inspection flap formed in said body by incising two substantially parallel cuts in the material of said body between said first and second faces thereof, each of said cuts extending inward from said first edge of said body toward said second edge, the material between said cuts forming said flap, said flap being liftable to expose an open slot between said cuts; and
a releasable flap securing strip with an adhesive layer that forms a repeatedly releasable and connectable adhesive bond with the material of said body, said flap securing strip disposed and connected to both said flap and said body on either side thereof, so as to allow said flap to be lifted for inspection and/or adjustment of an underlying catheter and then reconnected to re-establish protection of the catheter.

10. The device of claim 9, wherein said flap includes a window aperture formed in said flap between said cuts forming said flap, and window cover formed of a transparent material, said cover connected to said flap around said window aperture so as to provide a protective barrier to penetration through said window aperture while allowing visual inspection of the catheter without lifting said flap or removing the device from the patient.

11. A device for securing and protecting an IV catheter and associated tubing inserted through the skin of a human patient in a location on a patient's limb where the limb is substantially cylindrical, comprising,
a flexible body, formed of a thin pliable material, said body having a first face and a second face opposed to said first face across the thickness of said body, a first edge and a second edge generally opposed to said first edge, a first end and a second end generally opposed to said first end, said body being of a sufficient length between said first and second ends to extend across the width of the limb to which the device is to be connected with said first end of said body extending outwardly from the edge of the limb adjacent thereto, with said second end of said body extending outwardly from the opposite edge of the limb, the distance of extension of said first end of said body from the adjacent edge of the limb being less than the width of the limb between the two edges of the limb, and the distance of extension of said second end of said body from the opposite edge of the hand being less than the width of the limb;
a first adhesive band disposed on said second face of said body at said first end thereof, said first adhesive band extending across said first end between said first and second edges and inward a short distance toward said second end of said body, said first band of adhesive disposed such that when said body of the device is placed over a patient's limb with said first face received against the skin of the limb and said first end of said body is wrapped around the adjacent edge of the limb to the opposite side of the limb said first adhesive band is disposed on said face of said body such that is positioned outward of the skin of the limb; preventing said first adhesive band from contacting the skin; and
a second adhesive band disposed on said first face of said body at said second end thereof, said second adhesive band extending across said second end between said first and second edges and inward a short distance toward said first end of said body, said second band of adhesive disposed such that when said body of the device is placed over the patient's limb and said first end wrapped around the limb, said second adhesive band will be brought into contact with said second face of said body adjacent to said first end and form a releasable adhesive bond therewith, and said first adhesive band will be brought into contact with said first face of said body adjacent to said second end and form a releasable adhesive bond therewith, securing said body around the patient's limb without allowing adhesive material to contact the skin of the patient.

12. The device of claim 11, further comprising a window aperture formed in said body between said first and second edges and said first and second ends, said aperture positioned in said body so as to be disposed over the site of penetration of an IV catheter through the skin of a patient on the patient's limb with the device connected around the patient's limb, so as to permit visual inspection of the catheter without removing the device from the patient.

13. The device of claim 12, further comprising a window cover formed of a transparent material, said cover connected to said body around said window aperture so as to provide a protective barrier to penetration through said window aperture while allowing visual inspection of the catheter without removing the device from the patient.

14. The device of claim 13, wherein said window cover includes an adhesive material applied to the lower face of said window cover to be brought into contact with the patient's skin around the catheter and to form a mild and easily separable adhesive bond between said window cover and the skin of the patient.

15. The device of claim 11, further comprising,
a catheter inspection slot extending into said body from said first edge of said body toward said second edge, between said first end of said body and said second end of said body, said inspection slot disposed and dimensioned so as to overlie the penetration site of a catheter in a patient's limb; and
an inspection slot cover formed of a transparent material, said inspection slot cover releasably connected to said body around said inspection slot, to be positioned over the catheter insertion site, so as to allow visual inspection of the catheter without removing either the inspection slot cover or the complete device from the patient, and to allow said cover to be lifted to provide physical access to the catheter and then re-adhered to re-establish protection of the catheter without removing and replacing the entire device.

16. The device of claim 11,
a catheter inspection flap formed in said body by incising two substantially parallel cuts in the material of said body between said first and second faces thereof, each of said cuts extending inward from said first edge of said body toward said second edge, the material between said cuts forming said flap, said flap being liftable to expose an open slot between said cuts; and
a releasable flap securing strip with an adhesive layer that forms a repeatedly releasable and connectable adhesive bond with the material of said body, said flap securing strip disposed and connected to both said flap and said body on either side thereof, so as to allow said flap to be lifted for inspection and/or adjustment of an underlying catheter and then reconnected to re-establish protection of the catheter.

17. The device of claim 16, wherein said flap includes a window aperture formed in said flap between said cuts forming said flap, and window cover formed of a transparent material, said cover connected to said flap around said window aperture so as to provide a protective barrier to penetration through said window aperture while allowing visual inspection of the catheter without lifting said flap or removing the device from the patient.
